(19) United States
(12) Patent Application Publication
(10) Pub. No.: US 2024/0127017 A1
(43) Pub. Date: Apr. 18, 2024

(54) ANALYSIS APPARATUS AND ANALYSIS APPARATUS SUPPORT SYSTEM USING SAME

(71) Applicant: KYOTO ELECTRONICS MANUFACTURING CO., LTD., Kyoto-shi, Kyoto (JP)

(72) Inventors: Nobuaki WATANABE, Kyoto-shi (JP); Takashi MATSUKI, Kyoto-shi (JP); Hidenori MATSUOKA, Kyoto-shi (JP); Ikumi KUROSE, Kyoto-shi (JP)

(73) Assignee: KYOTO ELECTRONICS MANUFACTURING CO., LTD., Kyoto-shi, Kyoto (JP)

(21) Appl. No.: 18/275,666

(22) PCT Filed: Feb. 22, 2022
(86) PCT No.: PCT/JP2022/007156

§ 371 Date: Aug. 3, 2023

(30) Foreign Application Priority Data
Feb. 26, 2021 (JP) 2021-030847

(51) Int. Cl. G06K 7/14; G06F 8/61

(52) U.S. Cl. CPC G06K 7/1417; G06F 8/61

(57) ABSTRACT

The analysis apparatus according to the present invention is capable of setting parameters and user authentication, which do not require key input. When a specific analysis apparatus is set so as to measure a specific type of data, a plurality of required parameters are converted to data having a format suitable for a relay medium and the converted data is recorded in the relay medium. An importing unit imports the plurality of parameters from the relay medium. A restoration unit is used to obtain, from the imported parameters, each parameter in the pre-conversion format thereof. The individual obtained parameters are transferred to a setting unit and set to a required setting destination. This being the case, the replay medium is created according to the support from a management device of a manufacturer and a repeater.

### TECHNICAL FIELD

This invention relates to analysis apparatuses, and in particular, relates to analysis apparatuses that facilitate a setting operation before the measurement work.

### BACKGROUND ART

In case of the actual measurement work for obtaining a target physical quantity or a target stoichiometric amount by means of the analysis apparatus, it is necessary to set various kinds of parameters before the measurement work in order to operate the analysis apparatus normally. For instance, in the operation of oxidation-reduction titration for standardize the sodium sulfite, the following parameters must be set up in advance.
(1) The end point decision method: in case of the relation graph between the titration amount and the pH value, the end point is an intersection of a tangent extended line of the curve till the pH value raises up and another tangent extended line of the curve after the pH value raised, and the angle formed by the tangent extended line of the raising curve is set (e.g. 40 degree).
(2) The data sampling conditions: the data sampling potential (e.g. 999 mV), and the data sampling titration amount (e.g. each 0.1 mL).
(3) The control speed mode: the intermittent time (of the injection of titrant) (e.g. 15 s), the titration amount at one time (e.g. 0.1 mL), and the injection speed (e.g. 1 s/mL).
(4) The stirrer speed: rps (e.g. 2 rps)

There are various modes of using the analysis apparatus, and the mode depend on a type of the measurement. Therefore, the manufacturer has to ship out the analysis apparatus installing an application program suitable for the analysis apparatus and the type of measurement, however, the cloud computing technology is popular in recent years, so that the user can obtain the application program suitable for the type of measurement through the cloud system.

In addition, since the analysis object of the analysis apparatus also includes confidential matters of the user company using the apparatus, if the third party can see or obtain the data easily, such situation is not convenient for the user. Accordingly, the analysis apparatus is configured to have the user authentication unit in order to make the user authentication before the use of the analysis apparatus, and only the admitted person can use the analysis apparatus.

### CITATION LIST

### Patent Literature

None

### Non Patent Literature

None

### SUMMARY OF INVENTION

### Technical Problem

It is configured that the above-mentioned each type of setting items is described in an operation manual provided by the manufacturer side of the analysis apparatus, and the user side inputs required items from an input display of the analysis apparatus before starting the measurement work.

Such operation, however, is very troublesome operation for the user, and may induce user's incorrect input. In the unlikely event that there is an incorrect input, the measurement work will be invalidated.

Where the application program is obtained by means of the cloud system, there is a problem that the analysis apparatus should have the communication function. The communication function is provided to the apparatus, so that the product cost increases due to the manufacturing process. In case of using the communication function, the communication cost increases in the wireless communication, and the location of the apparatus is limited in the wired line communication.

In view of keeping the confidentiality of the data, it is necessary to make the user authentication before the measurement work. It is general that the user authentication is executed by inputting a user ID (including a group that the user belongs to) and a password. In usual, since the ID and the password are out of the user's recognition, he sometimes does not remember them when he starts the analysis apparatus, and he must input them by keyboard even if he remembers them. Such operation is very troublesome for the user.

The present invention is proposed in view of the above-mentioned problem in the conventional arts, and has an object to provide the analysis apparatus wherein the parameters and the user authentication can be set without using the keyboard and the application program can be set without the communication function.

### Solution to Problem

<Setting of Parameters>

The present invention is proposed in view of the above-mentioned conventional arts, and configured as follows.

In a relay medium, a plurality of parameters required when a specific type of measurement is performed on a specific analysis apparatus is converted to a data in a specific format suitable for the relay medium, and recorded therein. Under such assumption, the analysis apparatus is configured as follows.

An importing unit imports the recorded plurality of parameters from the relay medium.

The imported parameters are decoded to data in an original format before the conversion by a restoration unit, thereby each parameter is obtained. The obtained each parameter is given to a setting unit, and the setting unit sets the parameters obtained from the importing unit to each required setting address.

In such case, the relay medium is created according to the support of a repeater and a manufacturer management device.

First, information specifying the analysis apparatus and the type of measurement is transmitted from the repeater carried by a user to the manufacturer management device, and hereby the parameters are transmitted from the manufacturer management device to the repeater. The parameters have been converted into a format corresponding to the relay medium by the manufacturer management device. In the repeater, the parameters are records in the relay medium and then outputted by the output unit. Upon receipt of the outputted parameters, the importing unit of the analysis apparatus imports the contents recorded in the relay medium.

As the relay medium, a contactless IC chip or a two-dimensional code can be employed.

When the relay medium is the contactless IC chip, the importing unit is a reader for the contactless IC chip, and the output unit of the repeater is a writer for the contactless IC chip. When the relay medium is the two-dimensional code, the import unis is a camera for importing the two-dimensional code as an image, and the output unit of the repeater is a display for displaying the image of the two-dimensional code.

<Installation of Application Program>

It is possible to apply the present invention to an installation of an application program to be used for a specific type of measurement by the analysis apparatus.

In the relay medium, when a specific type of measurement is measured on a specific analysis apparatus, an application program for operating the analysis apparatus is converted to data in a specific format suitable for the relay medium and recorded therein.

The importing unit of the analysis apparatus imports the application program from the relay medium, and then, the restoration unit decodes the imported application program into a format before the conversion, thereby the application program in the original format is obtained. The installation unit installs the obtained application program.

In this case, the relay medium is also created according to the support of the repeater and the manufacturer management device.

The repeater includes a communication unit that transmits information specifying the analysis apparatus and the type of measurement to the manufacturer management device, and receives an application program for the analysis apparatus and for corresponding to the information specifying the analysis apparatus and the type of measurement transmitted from the manufacturer management device. A conversion unit of the repeater converts the application program to a specific format suitable for the relay medium, and then the output unit outputs the obtained application program in the specific format to the relay medium. Upon receipt of the outputted data, the importing unit of the analysis apparatus reads out the contents in the relay medium.

The manufacturer management device includes the communication unit, which receives the information specifying the analysis apparatus and the type of measurement transmitted from the repeater, and gives the information to a storage unit, and then transmits to the repeater the corresponding application program obtained from the storage unit. Besides, in this case, the conversion unit is located on the repeater side.

The repeater is configured to be provided with the format conversion unit in the above description. The conversion of the data to the format suitable for the relay medium may be executed before recording in the storage unit of the management device. In this case, the repeater does not need the format conversion unit.

In this case, the relay medium is a contactless IC chip or a two-dimensional code, and the output unit is a writer for the contactless IC chip or a display for displaying the two-dimensional code, and the importing unit is a reader for the contactless IC chip or a camera for importing the image of the two-dimensional code, according to the description of <Setting of Parameters>.

<User Authentication>

In the same as the above two examples, the relay medium records the user information showing the availability of the analysis apparatus and corresponding to a specific user or group in a specific data format suitable to the relay medium.

The importing unit of the analysis apparatus imports the user information recorded in the relay medium, the restoration unit decodes the imported user information in the specific format to the data in an original format before the conversion. The obtained user information is given to the user authentication unit. The user authentication unit decides, based on the user information thus obtained, whether or not the specific user or group can use the analysis apparatus.

In this case, the contactless IC chip and the two-dimensional code can be used as the relay medium, too. In general, the contactless IC chip and the two-dimensional code are realized by a form of the employee ID card or the union ID card issued by the specific company or organization.

In the present invention, the user authentication can be performed in a unique system without the employee ID card.

The analysis apparatus is provided with the user authentication unit that records a user or group able to (or not able to) use the analysis apparatus. The format conversion unit converts the user information to the data in the specific format suitable for the relay medium when the specific user or group is registered in the user registration unit. The output unit records the user information in the specific format obtained by the format conversion unit in the relay medium.

The recorded data in the repeater imported from the relay medium is imported by the importing unit of the analysis apparatus through the relay medium, which is given to the restoration unit. The restoration unit decodes the imported data in the specific format to the data before the conversion, and the user authentication unit decides whether or not the specific user or group can use the device.

The user authentication can be realized by the support of the repeater.

The repeater, that is provided with the importing unit, imports through the relay medium the user information outputted from the output unit of the analysis apparatus, and records the data in the storage unit. The output unit of the repeater outputs the data in the specific format recorded in the storage unit to the relay medium. Hereby, the importing unit of the analysis apparatus is operable to import the contents recorded in the relay medium, as described above.

Besides, also in this case, the contactless IC chip or the two-dimensional code can be used as the relay medium. In case of the contactless IC chip, the output unit (the writer) of the analysis apparatus can read the user information recorded in the relay medium from the importing unit (reader) of the analysis apparatus without the repeater.

### Advantageous Effects of Invention

As described above, without using the writing tools and the keyboard, the analysis apparatus is operable to set both setting items for operating the analysis apparatus and parameters corresponding thereto to required setting addresses. Thereby, the steps for the setting operation before starting the measurement work can be reduced, and the necessary items can be set without errors.

In the analysis apparatus without installing the application program required when the specific type of measurement is performed by the specific analysis apparatus, the application program for the specific type of measurement work is obtained from the manufacturer management device through the repeater, and recorded in the relay medium through the output unit of the relay medium, thereby the application program can be imported in the analysis apparatus.

In addition, there is an advantage that the user authentication can be simplified by converting and recording the user information to the data in the format suitable for the relay medium, and decoding the data to the original format before the use of the device.

### BRIEF DESCRIPTION OF DRAWINGS

FIG. 1 is a block diagram for setting parameters;

FIG. 2 is a block diagram for installing an application program; and

FIG. 3 is a block diagram for performing the user authentication.

### DESCRIPTION OF EMBODIMENTS

<Setting of Parameters>

When an actual measurement is performed for obtaining a target physical quantity or a target stoichiometric amount by means of a specific analysis apparatus, it is necessary to set various kinds of parameters in advance in order to operate the analysis apparatus normally, as described in a section of Background Art. In the present invention, at first, the parameters are converted to data in a format suitable for a two-dimensional code, and then recorded as a two-dimensional code.

It can be assumed that the two-dimensional code is printed on a sheet. For instance, it is general that the setting items corresponding to each type of measurement and parameter corresponding to each setting item are printed on a printed operation manual attached with the device at the purchase.

In recent years, instead of supplying the operation manual as a printed matter, the data of the operation manual is transmitted to a user via communication. In such case, the operation manual is located in the manufacturer's server or imported to a user's personal computer from the manufacturer's server. When the operation manual is printed out after imported to the user's personal computer, it is the same as the printed operation manual.

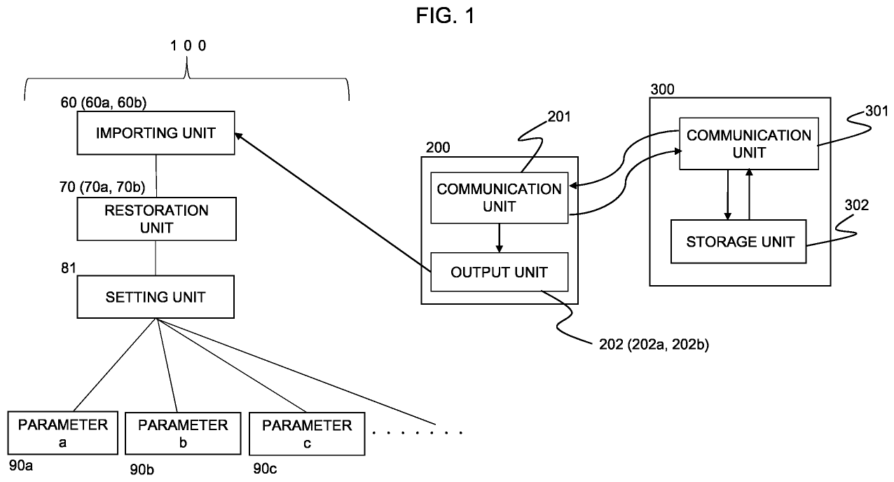

On the other hand, when the user does not have the operation manual on hand, the user can obtain it by accessing to a manufacturer management device 300 via communication unit 201 of a smartphone, a tablet terminal and so on (referred hereinafter to a repeater 200), as shown in FIG. 1.

It is assumed that the manufacturer converts the setting items for each measurement type executed by the analysis apparatus and the parameters for the setting items to the two-dimensional codes, and records the codes in the storage unit 302 of the manufacturer management device for every analysis apparatus.

When the information specifying the analysis apparatus and the measurement type to be executed are transmitted to the communication unit 301 of the manufacturer management device 300 from the communication unit 201 of the user's repeater 200, the management device 300 extracts the corresponding parameters from the storage unit 302 and transmits them to the user's repeater 200 via communication unit 301. The transmitted two-dimensional code is displayed on a display 202a as an output unit 202 of the repeater 200.

On the other hand, a camera 60a is provided to the analysis apparatus as an importing unit 60. The two-dimensional code, which is given to the user as the printed matter or displayed on the display 202a of the repeater 200, is recognized as an image by the camera 60a provided to the analysis apparatus, and given to a decoder 70a as a restoration unit 70. Upon receipt of the image of the two-dimensional code, the decoder 70a analyzes the image of the two-dimensional code and obtains the plurality of setting items and the corresponding parameters included in the image. The parameter corresponding to each setting item obtained by the decoder 70a is given to a setting unit 81. The setting unit 81 sets the each parameter corresponding to the setting item to a location 90(90a, 90b, . . . ) that is a location to be set the parameter corresponding to each setting item (otherwise, set on a specific address on the program).

According to the above process, the parameters necessary for the measurement are set to the specific location or address, whereby it is possible to start the measurement work without any troublesome setting operation.

### <Installation of Application Program>

There are various modes of using the analysis apparatus, which depend on a type of measurement. Accordingly, the manufacturer inevitably ships the analysis apparatus installing an application program corresponding to the analysis apparatus and the type of measurement. In these days, however, the cloud computing system becomes very popular, so that the application program corresponding to the type of measurement can be obtained through the cloud system.

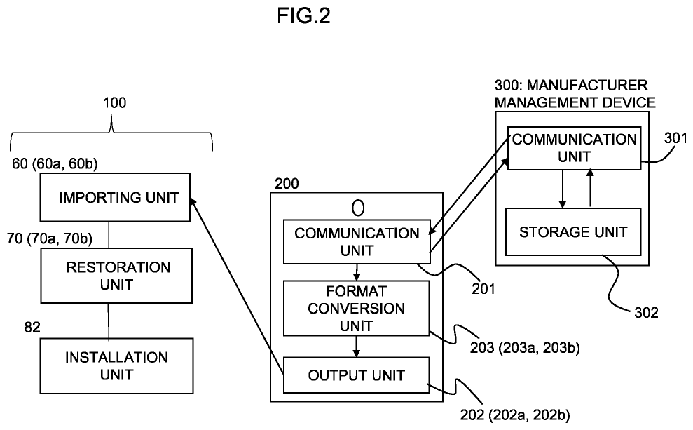

The present invention has a purpose that the cloud computing system is available for the analysis apparatus without the communication function. FIG. 2 is a diagram showing a system for realizing it.

First of all, the information for specifying the analysis apparatus and the information for specifying the type of the measurement to go on are transmitted from the repeater 200 to the manufacturer management device 300 by means of the communication unit 201.

The information for specifying the analysis apparatus and the information specifying the measurement type, that are received by a communication unit 301 (receiving unit) of the management device 300, are given to a storage unit 302. The storage unit 302, which stores application programs corresponding to the information for specifying the analysis apparatus and the information for specifying the measurement type, sends back the application program corresponding to them towards the repeater 200 through the communication unit 301 (sending unit).

The repeater 200, which obtained the specific application program via communication unit 201 (a receiving unit), is provided with an encoder 203a as a format conversion unit 203. The encoder 203a encodes the application program obtained as above to a two-dimensional code as a relay medium, which is displayed on a display 202a as an output unit 202.

The analysis apparatus 100 is provided with a camera 60a as an importing unit 60 and a decoder 70a as a restoration unit 70. The two-dimensional code displayed on the display 202a as described above is imported as an image to the analysis apparatus by the camera 60, and then decoded to the application program in an original format before the conversion by the decoder 70a.

The decoded application program is installed in a required location or a required address by an installation unit 82.

The repeater 200 is configured to have the format conversion unit 203 in the above description, however, when the manufacturer previously converts the application program to the data corresponding to the two-dimensional code or an IC chip before recording the application programs in the storage unit 302 of the repeater 300, it is possible to obtain the same effect as above. In such case, it is not necessary to provide the format conversion unit 203 to the repeater 200, and the format conversion unit may be installed in (or out of) the manufacturer management device 300.

Even in such case, it is necessary to set the parameters corresponding to the measurement type, however, the following method may be proposed. The parameters may be inputted by means of the display of the repeater when the application program is installed in the repeater, otherwise, after the program was installed in the repeater, the parameters corresponding to the setting items may be obtained from the manufacturer management device 300 through the repeater 200 in the same manner as above, and then converted and recorded as the two-dimensional code.

<User Authentication>

The two-dimensional code as the relay medium is allowed to record information referring to users of the analysis apparatus as well as the data with respect to the functions of the analysis apparatus as mentioned above. The user is not only an individual certainly, but also has a concept including groups such as a department and a team in the workplace. The user also includes information that “authorized” or “not authorized” a specific person (group) to use a specific function.

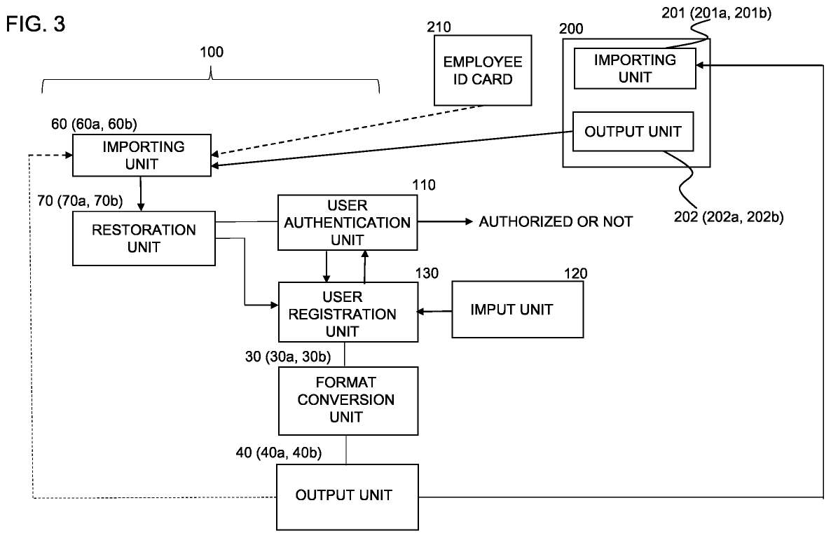

The person that belongs to a company carries an employee ID card issued by the company. The contents of the employee ID card are converted to the two-dimensional code, and printed on a part of the employee ID card, thereby the printed two-dimensional code is available to the use of the analysis apparatus. In case of displaying the two-dimensional code of the employee ID card on the display of the smartphone and the like, it is also available to the use of the device. FIG. 3 is a diagram showing a system for the user authentication by means of the two-dimensional code.

First, a user selects “USER REGISTRATION” by the input unit 120 of the analysis apparatus 100. In this state, the two-dimensional code of his employee ID card 210 is imported as an image through the camera 60a as the importing unit 60, and then decoded by the decoder 70a as the format restoration unit 70, whereby the items printed on the employee ID card are obtained and recorded in the recoding unit 130.

Here, it may use the printed two-dimensional code or the two-dimensional code displayed on the display of the smartphone, but the user may input necessary items such as a name, a belonging department, an ID, a password, and etc. from the input unit 120 by manual operation.

After the user registration is made in such way, the user, who wishes to user the analysis apparatus, selects “USER AUTHENTICATION” by means of the input means 120 before the actual measurement work, and makes the camera 60a import the two-dimensional code of the employee ID card. The information of the two-dimensional code is decoded by the decoder 70a, and given to the user authentication unit 110. The user authentication unit 110 compares the obtained items with the items recorded in the registration unit 130, and judges whether or not the user can use the analysis apparatus.

For the company issuing the employee ID card, the above method of the user authentication is enough, but there are many users belonging to the companies that do not issue the employee ID card. In such case, the analysis apparatus is required to make the user authentication in a unique way.

First, the user registration is completed by inputting the name, the belonging department, the user ID, the password, and etc. to the user registration unit 130 from the input unit 120, such as the keyboard.

When the user has to be authenticated before the measurement by the analysis apparatus, it is general that the user inputs the ID, and the password from the input unit 120. The user authentication, however, can be simplified, if the user is authenticated by using the two-dimensional code as described hereinafter.

When the user registration is completed, the user registration unit 100 displays an inquiry whether or not to issue the two-dimensional code in relevant to the user on the display 40a. After the user allows the issue of the two-dimensional code, the encoder 30a as the format conversion unit 30 starts up, and converts the information such as the specific user's ID, password, and so on recorded in the user registration unit 100 to the two-dimensional code, and displays it on the display 40a. In the user's repeater 200, the displayed two-dimensional code is imported as an image by means of the camera 201a as the importing unit 201, and recorded in the storage unit. Hereby, it is possible to display the two-dimensional code on the display unit 201 of the repeater 200 at any time. It is needless to say that the user may carry the two-dimensional code encoded by the encoder 30a as the printed matter.

The user has to make the user authentication before the actual measurement operation. At this time, the user displays the two-dimensional code recorded in the above-mentioned way on the display 202a as the output unit 202 of the repeater 200, and captures the image of the two-dimensional code (or the image of the two-dimensional code on the printed matter) by the camera 60a provided to the analysis apparatus, and then inputs the image to the decoder 70a. The decoder 70a inquires the decoded result to the user authentication unit 110, thereby the user authentication is completed.

<Case of Using Contactless IC Chip>

In any of the above-mentioned embodiments, a contactless IC chip is available as the relay medium instead of the two-dimensional code.

In this case, <setting of parameters> was performed as follows. Each parameter, which has been converted to the data in a format suitable for the contactless IC chip by the management device 300 of the manufacturer, is stored in advance in the storage unit 302. The writer 202b capable of writing the data (the parameters) into the contactless IC chip is employed as the output unit 202 of the repeater 200. The reader 60b capable of reading the contents of the contactless IC chip on which the parameters are written is employed as the importing unit 60 of the analysis apparatus.

In a step of <Installation of Application Program>, the encoder 203b is used as the format conversion unit 203 of the repeater 200, which converts the application program transmitted from the management device of the manufacturer into the format suitable for the contactless IC chip, and then writes it into the contactless IC chip through the writer 202b as the output unit 202. In this way, the application program written into the contactless IC chip is read by the reader 60b as the importing unit 60 of the analysis apparatus 100, and then decoded to the data in the original format before the conversion by the decoder 70b.

In the step of <User Authentication>, the user information is converted to the data in the format suitable for the contactless IC chip by the encoder 30b as the format conversion unit 30 provided to the analysis apparatus, and written into the contactless IC chip by the writer 40b as the output unit 40.

The data written into the contactless IC chip is read by the reader 201b as the importing unit of the repeater 200, and recorded in the contactless IC chip by the writer 202b as the output unit 202. The contents of the contactless IC chip, that may be carried by the user, if necessary, is recorded again in the contactless IC chip by the writer 202b as the output unit of the repeater 200 and then read by the reader 60b of the analysis apparatus 100.

In the analysis apparatus, the reader 60b as the importing unit 60 reads the contents recorded in the contactless IC chip, and gives the contents to the decoder 70b as the restoration unit 70, thereby the data is decoded to the data in the original format before the conversion.

Otherwise, as shown as a dot line in FIG. 3, it is possible to directly give the contents, which was recorded in the contactless IC chip by the writer 40b, to the reader 60b of the analysis apparatus 100 without using the repeater 200.

### INDUSTRIAL APPLICABILITY

As described above, the present invention is configured that the setting items for the analysis apparatus before operating the measurement in each type are converted to the two-dimensional code without using the writing tool and the keyboard, and set to the analysis apparatus before the measurement work, thereby it is possible to simplify the setting operation before the measurement work more than ever and eliminate the input errors. The invention is very effective.

Furthermore, since the data relevant to the user is converted to the two-dimensional code, the user authentication becomes easy and the process before the measurement work is performed speedy.

### REFERENCE SIGNS LIST

30 Format conversion unit
40 Display unit
60 Camera
70 Restoration unit
80 Setting unit
82 Installation unit
90 Parameter setting memory (Address)
100 Analysis apparatus
110 User authentication unit
120 Input unit
130 Registration unit
200 Repeater
201 Communication unit
202 Output unit
203 Format conversion unit
300 Management device
301 Communication unit
302 Storage unit

1. An analysis apparatus comprising:
an importing unit for importing a plurality of parameters, which is required when a specific type of measurement is performed on a specific analysis apparatus, from a relay medium recording the plurality of parameters converted in a specific format;
a restoration unit for decoding the imported parameters in the specific format to each parameter in an original format before the conversion; and
a setting unit for setting the parameters obtained by the restoration unit to each required setting address.

2. An analysis apparatus support system including an analysis apparatus, a repeater, and a manufacturer management device,
the analysis apparatus comprising:
an importing unit for importing a plurality of parameters, which is required when a specific type of measurement is performed on a specific analysis apparatus, from a relay medium recording the plurality of parameters converted in a specific format;
a restoration unit for decoding the imported parameters in the specific format to each parameter in an original format before the conversion;
a setting unit for setting the parameters obtained by the restoration unit to each required setting address,
the repeater comprising:
a communication unit for transmitting information specifying the analysis apparatus and the type of measurement to the manufacturer management device, and receiving data in the specific format corresponding to the information specifying the analysis apparatus and the type of measurement transmitted from the manufacturer management device;
an output unit for outputting the data in the specific format,
the manufacturer management device comprising:
a communication unit for receiving the information specifying the analysis apparatus and the type of measurement transmitted from the repeater, and giving the received information to a storage unit, and transmitting to the repeater the data in the specific format obtained from the storage unit; and
the storage unit for recording the data in the specific format corresponding to the information specifying the analysis apparatus and the type of measurement.

3. An analysis apparatus comprising:
an importing unit for importing an application program for operating the analysis apparatus, which is required when a specific type of measurement is performed on the analysis apparatus, from a relay medium recording the application programs converted in a specific format;
a restoration unit for decoding the imported application program to the application program in an original format before the conversion; and
an installation unit for installing the application program obtained by the restoration unit.

4. An analysis apparatus support system including an analysis apparatus, a repeater, and a manufacturer management device,
the analysis apparatus comprising:
an importing unit for importing an application program for operating the analysis apparatus, which is required when a specific type of measurement is performed on the analysis apparatus, from a relay medium recording the application program converted in a specific format suitable for the relay medium;
a restoration unit for decoding the imported application program in the specific format to the application program in an original format before the conversion;
an installation unit for installing the obtained application program,
the repeater comprising:
a communication unit for transmitting information specifying the analysis apparatus and the type of measurement to the manufacturer management device, and receiving the application program for operating the analysis apparatus and corresponding to the information specifying the analysis apparatus and the type of measurement transmitted from the manufacturer management device;
a conversion unit for converting the received application program into the specific format;
an output unit for outputting the obtained application program in the specific format to the relay medium,
the manufacturer management device comprising:
a communication unit for receiving the information specifying the analysis apparatus and the type of measurement transmitted from the repeater, and giving the received information to a storage unit, and transmitting the corresponding application program obtained from the storage unit to the repeater; and
the storage unit for giving the communication unit the application program corresponding to the information specifying the analysis apparatus and the type of measurement obtained from the communication unit.

5. An analysis apparatus support system including an analysis apparatus, a repeater, and a manufacturer management device,
the analysis apparatus comprising:
an importing unit for importing an application program for operating the analysis apparatus, which is required when a specific type of measurement is performed on the analysis apparatus, from a relay medium recording the application programs converted in a specific format suitable for the relay medium;
a restoration unit for decoding the imported application program in the specific format to the application program in an original format before the conversion;
an installation unit for installing the obtained application program,
the repeater comprising:
a communication unit for transmitting information specifying the analysis apparatus and the type of measurement to the manufacturer management device, and receiving from the manufacturer management device the application program in the specific format operating the analysis apparatus and corresponding to the information specifying the analysis apparatus and the type of measurement;
an output unit for outputting the obtained application program in the specific format to a relay medium,
the manufacturer management device comprising:
a communication unit for receiving the information specifying the analysis apparatus and the type of measurement transmitted from the repeater, and giving the received information to a storage unit, and transmitting the corresponding application program obtained from the storage unit to the repeater; and
the storage unit for giving the communication unit the application program in the specific format corresponding to the information specifying the analysis apparatus and the type of measurement obtained from the communication unit.

6. An analysis apparatus comprising:
an importing unit for importing user information showing a user availability of the analysis apparatus and corresponding to a specific user or group, from a relay medium recording the user information converted in a specific format;
a restoration unit for decoding the imported user information in the specific format to an original format before the conversion, and giving the obtained user information to an authentication unit; and
the authentication unit for deciding whether or not the specific user or group can use the analysis apparatus based on the obtained user information.

7. An analysis apparatus support system including an analysis apparatus and a repeater,
the analysis apparatus comprising:
a user registration unit for registering a user or a group able to use the analysis apparatus;
a format conversion unit, after a specific user or group is registered in the user registration unit, for converting information of the registered user into a specific format suitable for a relay medium;
an output unit for outputting, to a relay medium, the user information converted in the specific format obtained by the format conversion unit;
an importing unit for importing, through the relay medium, the user information outputted from an output unit of the repeater recording the user information in the specific format;
a restoration unit for decoding the user information in the specific format to the user information in an original format before the conversion based on the imported user information in the specific format;
a user authentication unit for deciding whether or not the specific user or group can use the analysis apparatus based on the user information obtained by the restoration unit;
the repeater comprising:
an importing unit for importing the user information outputted from the output unit of the analysis apparatus; and
a storage unit for recording data in the specific format; and
the output unit for outputting the data in the specific format recorded the in the storage unit to the relay medium.

8.-16. (canceled)

17. The analysis apparatus according to claim 1, wherein the relay medium is a contactless IC chip, and the importing unit is a reader for the contactless IC chip.

18. The analysis apparatus according to claim 3, wherein the relay medium is a contactless IC chip, and the importing unit is a reader for the contactless IC chip.

19. The analysis apparatus according to claim 1, wherein the relay medium is a two-dimensional code, and the importing unit is a camera.

20. The analysis apparatus according to claim 3, wherein the relay medium is a two-dimensional code, and the importing unit is a camera.

21. The analysis apparatus support system according claim 2, wherein the relay medium is a contactless IC chip, the importing unit of the analysis apparatus is a reader for the contactless IC chip, and the output unit of the repeater is a writer for the contactless IC chip.

22. The analysis apparatus support system according to claim 4, wherein the relay medium is a contactless IC chip, the importing unit of the analysis apparatus is a reader for the contactless IC chip, and the output unit of the repeater is a writer for the contactless IC chip.

23. The analysis apparatus support system according to claim 5, wherein the relay medium is a contactless IC chip, the importing unit of the analysis apparatus is a reader for the contactless IC chip, and the output unit of the repeater is a writer for the contactless IC chip.

24. The analysis apparatus support system according to claim 2, wherein the relay medium is a two-dimensional code, the importing unit of the analysis apparatus is a camera for importing the two-dimensional code as an image, and the output unit of the repeater is a display for displaying the two-dimensional code.

25. The analysis apparatus support system according to claim 4, wherein the relay medium is a two-dimensional code, the importing unit of the analysis apparatus is a camera for importing the two-dimensional code as an image, and the output unit of the repeater is a display for displaying the two-dimensional code.

26. The analysis apparatus support system according to claim 5, wherein the relay medium is a two-dimensional code, the importing unit of the analysis apparatus is a camera for importing the two-dimensional code as an image, and the output unit of the repeater is a display for displaying the two-dimensional code.

27. The analysis apparatus according to claim 6, wherein the relay medium is a contactless IC chip, and the importing unit is a reader for the contactless IC chip.

28. The analysis apparatus according to claim 6, wherein the relay medium is a two-dimensional code, and the importing unit is a camera.

29. The analysis apparatus support system according to claim 7, wherein the relay medium is a contactless IC chip, the importing unit of the analysis apparatus is a reader for reading data from the contactless IC chip, the output unit of the analysis apparatus is a writer for writing the data into the contactless IC chip, the importing unit of the repeater is a reader for reading the data from the contactless IC chip, and the output unit of the repeater is a writer for writing the data into the contactless IC chip.

30. The analysis apparatus support system according to claim 7, wherein the relay medium is a two-dimensional code, the importing unit of the analysis apparatus is a camera for importing the image of the two-dimensional code, the output unit of the analysis apparatus is a display for displaying the two-dimensional code, the importing unit of the repeater is a camera for importing the image of the two-dimensional code, and the output unit of the repeater is a display for displaying the two-dimensional code.

31. The analysis apparatus support system according to claim 7, wherein the relay medium is a contactless IC chip, the importing unit of the analysis apparatus is a reader for reading data from the contactless IC chip, the output unit of the repeater is a writer for writing the data into the contactless IC chip, and the reader directly reads contents of the contactless IC chip written by the writer.
